(19) United States
(12) Patent Application Publication
(10) Pub. No.: US 2009/0264772 A1
(43) Pub. Date: Oct. 22, 2009

(54) FASH FLUORESCENCE IMAGING DEVICE FOR DIFFUSE OPTICAL TOMOGRAPHY

(75) Inventors: Willen Peter Van Der Brug, Endhoven (NL); Martinus Bernardus Van Der Mark, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/375,053

(22) PCT Filed: Jul. 23, 2007
(86) PCT No.: PCT/IB2007/052916

§ 371 Date: Jan. 26, 2009

(30) Foreign Application Priority Data
Jul. 31, 2006 (EP) 06118181.4

(51) Int. Cl. A61B 6/00

(52) U.S. Cl. 600/476

(57) ABSTRACT

A device for imaging an interior of a turbid medium arranged to couple excitation light from a light source (5) into a receiving volume (20) containing a turbid medium from multiple entrance positions for light relative to the turbid medium (25) simultaneously. Multiple entrance positions for light may be created by coupling excitation light into the receiving volume (20) from M discrete entrance positions for light chosen from a plurality of N discrete entrance positions for light (M<=N) or by coupling excitation light into the receiving volume (20) from multiple entrance positions for light with at least a subset of the multiple entrance positions for light forming a continuum. An example of the latter option is the use of a spatially extended flash lamp.

The invention relates to a device for imaging an interior of a turbid medium comprising:

a) a receiving volume for accommodating the turbid medium;

b) a light source for emitting excitation light, with the excitation light chosen such that it causes fluorescent emission in a fluorescent agent in the turbid medium;

c) coupling means for optically coupling the light source to the receiving volume, with the coupling means comprising an entrance position for light from which to irradiate the receiving volume;

d) a photodetector unit for detecting fluorescence light emanating from the receiving volume as a result of the irradiation of the turbid medium with excitation light from the light source.

The invention also relates to a method for imaging an interior of a turbid medium comprising the following steps:

a) emission of excitation light from a light source, with the excitation light chosen such that it causes fluorescent emission in a fluorescent agent in the turbid medium;

b) optically coupling the light source to the receiving volume for irradiating the receiving volume with excitation light from the light source from an entrance position for light;

c) detection of fluorescence light emanating from the receiving volume as a result of the irradiation of the turbid medium with excitation light from the light source.

The invention also relates to a medical image acquisition device comprising:

a) a receiving volume for accommodating a turbid medium;

b) a light source for emitting excitation light, with the excitation light chosen such that it causes fluorescent emission in a fluorescent agent in the turbid medium;

c) coupling means for optically coupling the light source to the receiving volume, with the coupling means comprising an entrance position for light from which to irradiate the receiving volume;

d) a photodetector unit for detecting fluorescence light emanating from the receiving volume as a result of the irradiation of the turbid medium with excitation light from light source.

An embodiment of a device of this kind is described in European patent application, with application number 05111164.9 (PH004270 attorney reference). The described device can be used for imaging an interior of a turbid medium, such as biological tissues. In medical diagnostics the method may be used for imaging an interior of a female breast. The receiving volume receives a turbid medium, such as a breast. Excitation light from a light source is coupled into the receiving volume from an entrance position for light, with the entrance position for light successively chosen from a plurality of entrance positions for light located at different positions relative to the turbid medium. The excitation light is chosen such that it causes fluorescent emission in a fluorescent agent in the turbid medium. Fluorescence light emanating from the receiving volume as a result of irradiating the turbid medium with excitation light is collected at a plurality of collection positions and used to derive an image of an interior of the turbid medium. Alternatively, if the light source emits light chosen such that it does not cause fluorescent emission in the turbid medium, collected light having passed through the turbid medium may be used to derive an image of an interior of the turbid medium. Typically, light having a wavelength within the range of 400 nm to 1400 nm is used for this purpose. In the following text, the latter procedure is called transillumination.

It is a drawback of the described device that fluorescence imaging is slow. In medical diagnostics, where the device may be used to image an interior of a female breast, long measurement times are uncomfortable for patients, increase the chance of patients moving during a measurement thus reducing imaging quality, and make imaging of processes or events on relatively small timescales hard. Within the context of imaging tumors in breast tissue, processes or events on relatively small timescales are, for instance, processes or events in which the heart cycle plays an important role, such as the dynamics of the blood flow over a systolic/diastolic cycle.

It is an object of the invention to reduce measurement times.

According to the invention this object is realized in that the device according to the opening paragraph is arranged to simultaneously couple excitation light from the light source into the receiving volume from multiple entrance positions for light relative to the turbid medium.

The invention is based on the recognition that although the position of an entrance position for light relative to the turbid medium is very important for image reconstruction when using the transillumination procedure, it is less relevant for fluorescence measurements. This recognition is based on the fact that the fluorescence light from the fluorescent agent in the turbid medium forms a secondary source of light relative to the primary light source emitting excitation light. Consequently, the fluorescent agent marks the primary source of the fluorescence light. As the position of an entrance position for light relative to the turbid medium is less relevant for fluorescence measurements, it is possible to simultaneously couple excitation light from the light source into the receiving volume from multiple entrance positions for light relative to the turbid medium instead of from one entrance position for light. Hence, the amount of excitation light is increased as compared to the situation in the known device. Consequently, more fluorescence light is generated in a fluorescent agent, if a sufficient amount of fluorescent agent is present, which in turn results in shorter measurement times while obtaining the same amount of fluorescence light as compared to the situation in the known device. The invention is particularly useful if the distribution of the fluorescent agent is localized in separate regions, which is the case for so-called targeted fluorescent agents.

It is an additional advantage of the invention that an increase in the amount of fluorescence light allows tracking of time-dependent fluorescent events varying on timescales that defied tracking up until now. In medical diagnostics, where the device may be used for, for instance, imaging an interior of a female breast, fast changing fluorescent events may occur in applications in which the heart cycle plays an important role. Increasing the amount of fluorescence light generated in a fluorescent agent reduces the time needed to obtain a sufficient amount of signal. Hence tracking of time-dependent fluorescent events becomes possible on smaller timescales than was possible before.

It is a further additional advantage of the invention that simultaneously coupling excitation light from the light source into the receiving volume from multiple entrance positions for light relative to the turbid medium also makes it possible to increase the amount of excitation light that is coupled into the receiving volume as compared to the situation in which excitation light is coupled into the receiving volume from a single entrance position for light without exceeding the maximum permissible exposure. In medical diagnostics, where the device may be used for imaging an interior of a female breast, the maximum permissible exposure limits the amount of energy that may be coupled into human skin per unit of area and per unit of time. However, in order to increase the amount of fluorescence light emanating from a breast or to image processes or events on relatively small timescales it is desirable to increase the amount of excitation light that is coupled into the breast. According to the invention, this is possible without exceeding the maximum permissible exposure by coupling excitation light into the receiving volume from multiple entrance positions for light relative to the turbid medium, because excitation light emanating from different entrance positions for light illuminates different areas of the turbid medium.

An embodiment of the device according to the invention is characterized in that the device is arranged for simultaneously coupling excitation light into the receiving volume from a first plurality of M discrete entrance positions for light chosen from a second plurality of N discrete entrance positions for light, wherein M is smaller than or equal to N, i.e. M≦N. This embodiment has several advantages. First of all, it allows the time necessary for a measurement to be reduced by a factor M as compared to the situation in the known device. Second, this embodiment allows to improve the signal to noise ratio in combination with a reduction in measurement time. However, in the latter case the reduction in measurement time is less than a factor M. Third, this embodiment allows tracking of a fast changing fluorescent event. As explained, in the known device excitation light is coupled into the receiving volume via a single entrance position for light. By coupling excitation light from the light source into the receiving volume from multiple entrance positions for light relative to the turbid medium simultaneously, the amount of fluorescence light emanating from the receiving volume is increased as compared to the situation in the known device. The increase in the number of entrance positions for light may be achieved by coupling multiple entrance positions for light to a single light source, by coupling each entrance position for light to its own individual light source, or a combination of these two possibilities. This increase in the amount of fluorescence light may be used to reduce measurement times, to improve the signal to noise ratio, possibly in combination with a reduction of measurement times, or to track a fast changing fluorescent event. Tracking of a fast changing fluorescent event becomes possible as the time needed to obtain a certain amount of signal is reduced.

A further embodiment of the device according to the invention is characterized in that the light source is arranged to emit light at multiple wavelengths simultaneously and wherein the coupling means are arranged to couple light of a single wavelength to at least one entrance position for light. As has been explained, the known device comprises a light source for emitting excitation light. As has also been explained, the known device may further comprise a further light source for emitting light capable of propagating through the turbid medium. This further light source may in fact be arranged to emit light at multiple wavelengths simultaneously. This light may be used to cause fluorescent emission in a fluorescent agent in the turbid medium albeit with a possibly lower fluorescence efficiency than would be possible with light chosen especially for the purpose of causing fluorescent emission in the fluorescent agent. However, use of such a further light source may be fully adequate for fluorescence imaging purposes. This embodiment, therefore, has the advantage that no extra light sources are required to implement the invention as compare to the known device when used for such transillumination purposes.

A further embodiment of the device according to the invention is characterized in that at least a subset of the multiple entrance positions for light forms a continuum. A single spatially extended light source, such as a flash lamp, with which the receiving volume may be irradiated from multiple entrance positions for light relative to the turbid medium that form a continuum, may be used to cause fluorescent emission in a fluorescent agent in the turbid medium. The shape of this light source may be chosen such that an emission surface of the light source facing the turbid medium and through which light is emitted has a shape that corresponds to the shape of a surface of the turbid medium facing the emission surface of the light source. In medical diagnostics, where the known device may be used for imaging an interior of a female breast, and extended light source may, for instance, be curved around a breast accommodated inside the receiving volume. This embodiment has the advantage that it allows for faster acquisition of fluorescence imaging data. This embodiment may be used in combination with other embodiments of the invention according to which discrete entrance positions for light may be used.

According to the invention the object of the invention to reduce measurement times is further realized in that the method according to the invention is adapted such that the step of optically coupling the light source to the receiving volume comprises optically coupling the light source to the receiving volume for irradiating the receiving volume with excitation light from the light source from multiple entrance positions for light simultaneously.

According to the invention this object is further realized in that the medical image acquisition device is arranged to simultaneously couple excitation light from the light source into the receiving volume from multiple entrance positions for light relative to the turbid medium.

These and other aspects of the invention will be further elucidated and described with reference to the drawings, in which:

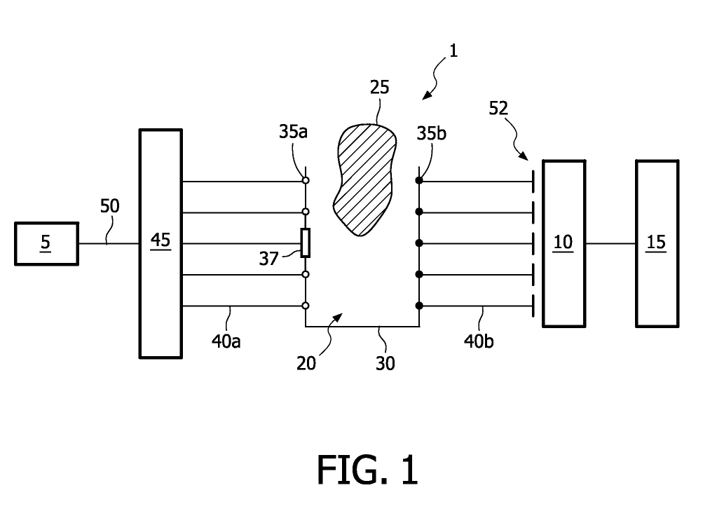

FIG. 1 schematically shows an embodiment of a device for imaging an interior of a turbid medium;

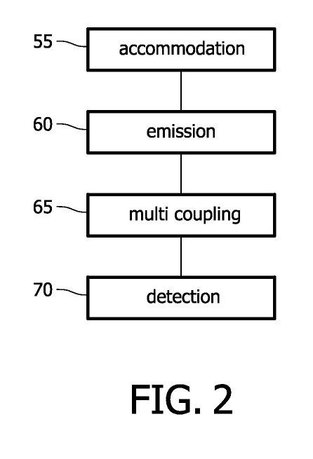

FIG. 2 shows a flow chart explaining the method according to the invention;

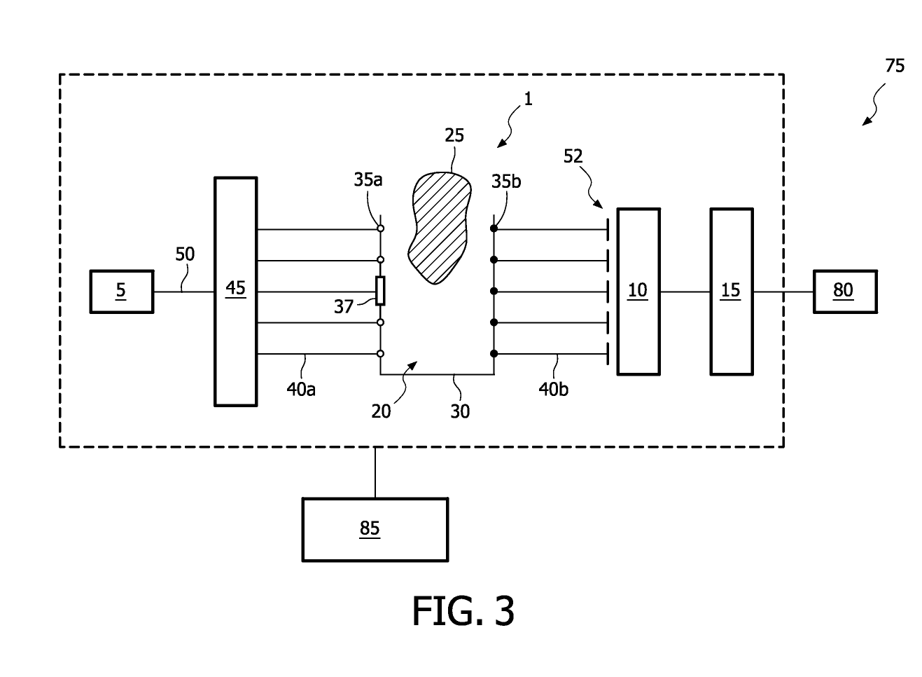

FIG. 3 shows an embodiment of a medical image acquisition device according to the invention.

FIG. 1 schematically shows an embodiment of a device for imaging an interior of a turbid medium. The device 1 includes a light source 5 for emitting excitation light with the excitation light chosen such that it causes fluorescent emission in a fluorescent agent in a turbid medium 25, a photodetector unit 10, an image reconstruction unit 15, a receiving volume 20 for receiving the turbid medium 25, said receiving volume 20 being bound by a receptacle 30, said receptacle comprising a plurality of entrance positions for light 35a and exit positions for light 35b, and light guides 40a and 40b coupled to said entrance positions for light 35a and exit positions for light 35b, respectively. The device 1 further includes a selection unit 45 for coupling the input light guide 50 to a number of selected entrance positions for light 35a in the receptacle. For the sake of clarity, entrance positions for light 35a and exit positions for light 35b have been positioned at opposite sides of the receptacle 30. In reality, however, they may be distributed around the receiving volume 20. If the device 1 is used for fluorescence measurements, the device 1 must be arranged such that excitation light emanating from the receiving volume 20 can be distinguished from fluorescence light emanating from the receiving volume 20. This can be achieved, for instance, by arranging the device 1 such that light emanating from the receiving volume 20 passes through an optical filter 52 that filters out excitation light. A turbid medium 25 is accommodated inside the receiving volume 20. According to one embodiment of the invention, the turbid medium 25 is then irradiated with excitation light from the light source 5 from a plurality of entrance positions for light 35a by simultaneously coupling the light source 5 using the selection unit 45 to a number of selected entrance positions for light 35a, said selected entrance positions for light being selected from the plurality of entrance positions for light 35a. This can be done by, for instance, simultaneously coupling excitation light into the receiving volume from a first plurality of M discrete entrance positions for light 35a chosen from a second plurality of N discrete entrance positions for light 35a, wherein M is smaller than or equal to N (M≦N). The excitation light emitted by the light source 5 is chosen such that it causes fluorescent emission in a fluorescent agent in the turbid medium 25. This embodiment enables to couple more excitation light from the light source 5 into the turbid medium 25 than would be the case if the turbid medium 25 were to be irradiated with excitation light from only a single entrance position for light without exceeding the maximum permissible exposure. In medical diagnostics, where the device 1 may be used for imaging an interior of a female breast, the maximum permissible exposure limits the amount of energy that may be coupled into, for instance, human skin per unit of area per unit of time. As a result, the embodiment also enables to image time-dependent events, such as events in which the heart cycle over a systolic and diastolic cycle plays an important role. Fluorescence light emanating from the receiving volume 20 is detected from a plurality of positions using the plurality of exit positions for light 35b and using photodetector unit 10. The image reconstruction unit 15 then uses the detected fluorescence light to derive an image of an interior of the turbid medium 25. According to a further embodiment of the invention the light source 5 may be further arranged such that the turbid medium 25 can be irradiated with light of multiple wavelengths, with the multiple wavelengths chosen such that this light is capable of propagating through the turbid medium 25. Although light having such a wavelength might not be optimal for exciting a fluorescent agent present in the turbid medium 25, such light may still cause sufficient fluorescence in the fluorescent agent. In that case the embodiment has the advantage that it is easy to implement as devices such as the device 1 usually already comprise a light source arranged to emit light of multiple wavelengths. According to a further embodiment of the invention, a spatially extended light source 37 may be used to irradiate the receiving volume 20 instead of, or in addition to at least one discrete entrance position for light 35a. The spatially extended light source 37 irradiates a receiving volume 20 from multiple entrance positions for light within the spatially extended light source 37 and with the multiple entrance positions for light forming a continuum. According to this embodiment a substantial part of the turbid medium 25 can be irradiated with excitation light enabling speeding up measurements.

FIG. 2 shows a flow chart explaining the method according to the invention. In step 55 the turbid medium 25 is accommodated in the receiving volume 20. In step 60 light source 5 emits excitation light, with the excitation light chosen such that it causes fluorescent emission in a fluorescent agent in the turbid medium 25. In step 65 the light source 5 is optically coupled to the receiving volume 20 for irradiating the receiving volume 20 with excitation light from the light source 5. According to the invention the light source 5 is optically coupled to the receiving volume 20 such that the receiving volume 20 can be irradiated with excitation light 5 from multiple entrance positions for light 35a simultaneously. This can be done by, for instance, optically coupling the light source 5 to the receiving volume 20 such that excitation light is coupled into the receiving volume 20 from a first plurality of M discrete entrance positions for light 35a chosen from a second plurality of N discrete entrance positions for light 35a, wherein M is smaller than or equal to N (M≦N). Another option is that the light source 5 may be arranged such that the turbid medium 25 can be irradiated with light of multiple wavelengths, with light of a single wavelength being coupled to at least one entrance position for light 35a and with the multiple wavelengths chosen such that this light is capable of propagating through the turbid medium 25. Although light having such a wavelength might not be optimal for exciting a fluorescent agent present in the turbid medium 25, such light may still cause sufficient fluorescence in the fluorescent agent. In that case the embodiment has the advantage that it is easy to implement as devices such as the device 1 usually already comprise a light source arranged to emit light of multiple wavelengths. A further option is the use of a spatially extended light source 37 to irradiate the receiving volume 20 instead of, or in addition to at least one discrete entrance position for light 35a. The spatially extended light source 37 irradiates a receiving volume 20 from multiple entrance positions for light within the spatially extended light source 37 and with the multiple entrance positions for light forming a continuum. According to this embodiment a substantial part of the turbid medium 25 can be irradiated with excitation light enabling speeding up measurements. In step 70 fluorescence light emanating from the receiving volume 20 as a result of the irradiation of the turbid medium 25 with excitation light from the light source 5 is detected. In order to only detect fluorescence light emanating from the receiving volume 20 the device 1 must be arranged such that excitation light emanating from the receiving volume 20 can be distinguished from fluorescence light emanating from the receiving volume 20. This can be achieved by, for instance, having an optical filter 52 that rejects excitation light but lets fluorescence light pass anywhere in the light path between the receiving volume 20 and the photodetector unit 10.

FIG. 3 shows an embodiment of a medical image acquisition device 75 according to the invention. The medical image acquisition device 75 comprises the device 1 discussed in FIG. 1 indicated by the dashed square. In addition to the device 1 the medical image acquisition device 75 further comprises a screen 80 for displaying an image of an interior of the turbid medium 25 reconstructed by the image reconstruction unit 15 and an input interface 85, for instance, a keyboard enabling an operator to interact with the medical image acquisition device 75.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A device for imaging an interior of a turbid medium comprising:
a) a receiving volume (20) for accommodating the turbid medium (25);
b) a light source (5) for emitting excitation light, with the excitation light chosen such that it causes fluorescent emission in a fluorescent agent in the turbid medium (25);
c) coupling means for optically coupling the light source (5) to the receiving volume (20), with the coupling means comprising an entrance position for light (35a) from which to irradiate the receiving volume (20);
d) a photodetector unit (15) for detecting fluorescence light emanating from the receiving volume (20) as a result of the irradiation of the turbid medium (25) with excitation light from the light source (5),
characterized in that
the device (1) is arranged to simultaneously couple excitation light from the light source (5) into the receiving volume (20) from multiple entrance positions for light (35a) relative to the turbid medium (25).

2. A device as claimed in claim 1, wherein the device is arranged for simultaneously coupling excitation light into the receiving volume (20) from a first plurality of M discrete entrance positions for light (35a) chosen from a second plurality of N discrete entrance positions for light (35a), wherein M is smaller than or equal to N, i.e. M≦N.

3. A device as claimed in claim 2, wherein the light source (5) is arranged to emit light at multiple wavelengths simultaneously and wherein the coupling means are arranged to couple light of a single wavelength to at least one of the entrance positions for light (35a).

4. A device as claimed in claim 1, wherein at least a subset (37) of the multiple entrance positions for light (35a) forms a continuum.

5. A method for imaging an interior of a turbid medium (25) comprising the following steps:
a) emission of excitation light from a light source (5), with the excitation light chosen such that it causes fluorescent emission in a fluorescent agent in the turbid medium (25);
b) optically coupling the light source (5) to the receiving volume (20) for irradiating the receiving volume (20) with excitation light from the light source (5) from an entrance position for light (35a);
c) detection of fluorescence light emanating from the receiving volume (20) as a result of the irradiation of the turbid medium (25) with excitation light from the light source (5),
characterized in that
the step of optically coupling the light source (5) to the receiving volume (20) comprises optically coupling the light source (5) to the receiving volume (20) for irradiating the receiving volume (20) with excitation light from the light source (5) from multiple entrance positions for light (35a) simultaneously.

6. A medical image acquisition device comprising:
a) a receiving volume (20) for accommodating a turbid medium (25);
b) a light source (5) for emitting excitation light, with the excitation light chosen such that it causes fluorescent emission in a fluorescent agent in the turbid medium (25);
c) coupling means for optically coupling the light source (5) to the receiving volume (20), with the coupling means comprising an entrance position for light from which to irradiate the receiving volume (20);
d) a photodetector unit (15) for detecting fluorescence light emanating from the receiving volume (20) as a result of the irradiation of the turbid medium (25) with excitation light from the light source (5)
characterized in that
the device is arranged to simultaneously couple excitation light from the light source (5) into the receiving volume (20) from multiple entrance positions for light relative to the turbid medium (25).
